(19) United States
(12) Patent Application Publication
(10) Pub. No.: US 2007/0128257 A1
(43) Pub. Date: Jun. 7, 2007

(54) Animal wipes having catnip

(76) Inventors: John Manolas, Lake Forest, IL (US); Elli Manolas, Lake Forest, IL (US)

Correspondence Address: Michael S. Leonard;EVEREST INTELLECTUAL PROPERTY LAW GROUP, P.O. Box 708, Northbrook, IL, 60065, US

(21) Appl. No.: 11/607,640

(22) Filed: Dec. 1, 2006

Related U.S. Application Data
(60) Provisional application No. 60/741,801, filed on Dec. 2, 2005.

(51) Int. Cl. A61K 36/53; A61K 9/70

(52) U.S. Cl. 424/443; 424/745

(57) ABSTRACT

An animal wipe or pet wipe is an applicator which carries a formulation containing catnip. One or more wipe is contained in a container and dispensed from the container. The wipe having catnip is rubbed on a pet, such as a cat.

### CROSS-REFERENCE TO RELATED APPLICATIONS

This application is a continuation of provisional application Ser. No. 60/741,801, filed Dec. 2, 2005.

### BACKGROUND OF THE INVENTION

The present invention generally pertains to animal wipes. More specifically, the present invention pertains to animal wipes having catnip. The present invention also pertains to methods of wiping animals, methods of providing catnip to animals, and methods of making animal wipes having catnip. The present invention is particularly useful for pets, for example, cats.

Currently, pet wipes are available on the market. Some are specific for cats, others for dogs, while some are for use on both. These wipes contain a water-based formulation that typically is comprised of the following ingredients.

| Ingredient | Use |
|---|---|
| Water | Carrier |
| Surfactant(s) | Wetting agent, oil emulsifier, cleaning |
| Vitamin E | Organic moisturizing & conditioning agent |
| Aloe | Organic moisturizing & conditioning agent |
| Jojoba Oil | Organic moisturizing & conditioning agent |
| Glycerin | Organic moisturizing & conditioning agent |
| Fragrance | To make product more attractive |
| Lanolin | Organic moisturizing & conditioning agent |
| Oatmeal | Organic moisturizing & conditioning agent |
| Wheat Germ Oil | Organic moisturizing & conditioning agent |
| Conditioner | Conditions fur, coat, and skin |
| Ethanol | Solubilizer and cleaning agent |
| Preservative | Prevents bacteria growth in formula |

Other ingredients can be used for similar purposes. Wipes are used as a bath alternative or in between baths for the animal. They are advertised as a way to keep the animal clean, smelling fresh, minimizing dander and shedding. For cats, the added advantage is that it reduces hairballs that cats tend to vomit. They tend to clean themselves and swallow loose hair. The hair accumulates, as it is difficult to digest. It either comes out in the form of vomit or in their stool. Cats have been known to get quite ill from hairballs. It is a cause of distress to them and to their owners.

Needs exist to improve animal wipes and provide new animal wipes for the reasons mentioned above and for other reasons.

### BRIEF DESCRIPTION OF THE FIGURES

FIG. 1 shows a container having animal wipes having catnip according to the present invention.

FIG. 2 is a view of the container of FIG. 1 showing a cap opened and an animal wipe having catnip being dispensed from the container.

FIG. 3 is a view of the container of FIG. 1 with a top removed and a role of animal wipes having catnip removed from the container.

FIG. 4 shows another container having animal wipes having catnip according to the present invention.

FIG. 5 is a view of the container of FIG. 4 showing a cap opened and an animal wipe having catnip being dispensed from the container.

FIG. 6 is a view of the container of FIG. 4 with a top removed and a role of animal wipes having catnip removed from the container.

### DETAILED DESCRIPTION OF THE INVENTION

In an embodiment, the present invention is a cat wipe with the addition of an ingredient that gives cats pleasure, catnip. Catnip is an herb known as Nepeta Cataria. It is in the mint family. Catnip is native to Europe and imported to the United States. It is now a very common weed in the United States. Cats are attracted to the scent of catnip.

Additional information about catnip:
Chemical that causes reaction is known as Nepetalactone
Not known exactly what happens in the cat's brain
Reaction to catnip is inherited as a dominant gene
Older cats and younger kittens seem to be less likely to react to it
Reaction typically lasts for a few minutes then the cat acclimates to it
Takes a couple of hours to reset so cat can get another reaction to it
Catnip is non-toxic to pets
Inhaled by cat to illicit response

In an embodiment, the present invention incorporates an amount of catnip into a liquid formulation that is applied to a wipe (for example, a baby wipe type packaging) and used on a cat. As cats do not typically like water or baths, the addition of catnip attracts the cat to the wipe and makes it easier for the owner to wipe the cat. Wiping the cat on a regular basis reduces cat dander (an allergen), reduces the amount of loose hair that could end up causing hair balls, and keeps the cat smelling fresh and looking clean.

A wide range of amounts of catnip can be suitable for the present invention. For example, embodiments of the invention may have formulations having about 0.000000001% to about 75% catnip. The present invention contemplates any lower and upper endpoints of the range. The catnip can be in various forms, including steeped or dissolved, oil and combinations thereof.

Examples of the ranges of ingredients for some formulations of the present invention are as follows:

| | | A Preferred |
|---|---|---|
| Ingredient | Percentage Range | Range |
| Water Phase | | |
| Water (soft or de-ionized) | To 100% | To 100% |
| Cat Nip (steeped or dissolved) | 0.000000001 to 75% | 0.001 to 10% |
| Surfactant | 0.00001 to 20% | 0.01 to 2.5% |
| Preservative | 0.0000001 to 20% | 0.01 to 2.5% |
| Aloe Vera | 0.0000001 to 20% | 0.01 to 2.5% |
| Lanolin | 0.0000001 to 20% | 0.01 to 2.5% |
| Oatmeal | 0.0000001 to 20% | 0.01 to 5% |
| Herbal Extracts | 0.0000001 to 20% | 0.01 to 5% |
| Oil Phase | | |
| Ethanol | 0.00001 to 20% | 0.01 to 5% |
| Vitamin E | 0.000001 to 20% | 0.01 to 2.5% |
| Wheat Germ Oil | 0.000001 to 20% | 0.01 to 2.5% |
| Vitamin A | 0.000001 to 20% | 0.01 to 25% |
| Surfactant | 0.000001 to 20% | 0.01 to 2.5% |

One method of making an embodiment of the present invention is to pre-mix the ingredients from the water phase and the oil phase separately. Add the oil phase to the water phase slowly under agitation. Depending on the type of towel (for example wipe or other applicator) the size of the towel and the quantity of towels in the container a certain amount of the above liquid will be added to each container to moisten the towels. This can be adjusted depending on the packaging of the product.

The preferred surfactant will be naturally derived (corn, coconut, palm, or sugar). Some examples are: Cocoamido Propyl Betaine, Plantarens, Sodium Lauryl Sulfates, Sodium Lauryl Ether Sulfates, Sodium Ether Sulfates, etc. Petroleum based surfactants are also suitable for use in the invention. Non-Ionic, Cationic, Anionic and Amphoteric surfactants can be used in virtually any combination.

Herbal extracts can include Chamomile, Cocoa Butter, Melaleuca, Rosemary, Soap Bark, and Yucca.

Other ingredients can be included if one wishes. For example, the ingredients used in existing pet wipes can be included in the present invention. Other ingredients also include Jojoba Oil, other herbal extracts, Fragrance (essential oil), Glycerin, Conditioners, Baking Soda, Proteins, Other Vitamins, etc.

In broad embodiments, the present invention pertains to catnip in virtually any liquid, gel or other form that is incorporated with or carried by a wipe or other applicator.

One preferred formulation of the present invention is:

| Ingredient | Percentage |
|---|---|
| Water Phase | |
| Water (soft) | To 100% |
| Cat Nip (steeped or dissolved) | 0.5% |
| Colesoft CAB (Cocoamido Propyl Betaine) | 0.2% |
| Oatmeal | 0.1% |
| Lanolin | 0.1% |
| Actiphyte of Chamomile | 0.05% |
| Actiphyte of Yucca | 0.05% |
| Methyl Paraben (preservative) | 0.1% |
| Propyl Paraben (preservative) | 0.3% |
| Oil Phase | |
| Ethanol | 0.5% |
| Vitamin E | 0.1% |
| Vitamin A | 0.1% |
| Wheat Germ Oil | 0.05% |
| Colesoft CAB (Cocoamido Propyl Betaine) | 0.3% |

There are equivalent ingredients for virtually all of the above-mentioned raw materials and any formulation components identified in this disclosure. One could use Isopropyl alcohol in place of ethanol; there are various trade names for the Colesoft CAB but they are all equivalent to Cocoamido Propyl Betaine; other preservatives are available for use such as glydant and phenonip.

According to the present invention, a formulation includes catnip and may contain any individual component or any combination of components identified in this disclosure. Also, the amounts of any of the components can be changed to any amount or range of amount as desired.

A wide variety of wipes, towels, towelettes, and other applicators are suitable for use with the present invention. Different sizes can be used depending on the embodiment, intended use, etc. For example, smaller sizes may be suitable for the independent pet stores, larger sizes for the superstores like Petsmart and Petco. The present invention can be embodied in an individual wipe, for sample purposes, for example; individual wipes packaged in a box (for example, between 10 and 50 individual wipes); a soft, re-sealable pack of wipes, such as 10 to 15 wipes, a canister of multiple wipes, such as 50 or more towelettes, with a pull through top. One or more wipes can be contained in a closable or a non-closable package. Of course, any other suitable packaging can be used with the present invention.

The animal wipes can be made of any material as desired and suitable for use with animals and pets. For example, the wipes could be a cloth. The cloth may be made of natural materials, synthetic materials or combinations of natural and synthetic materials.

The wipes or applicators may be smooth or textured or have other surfaces as desired.

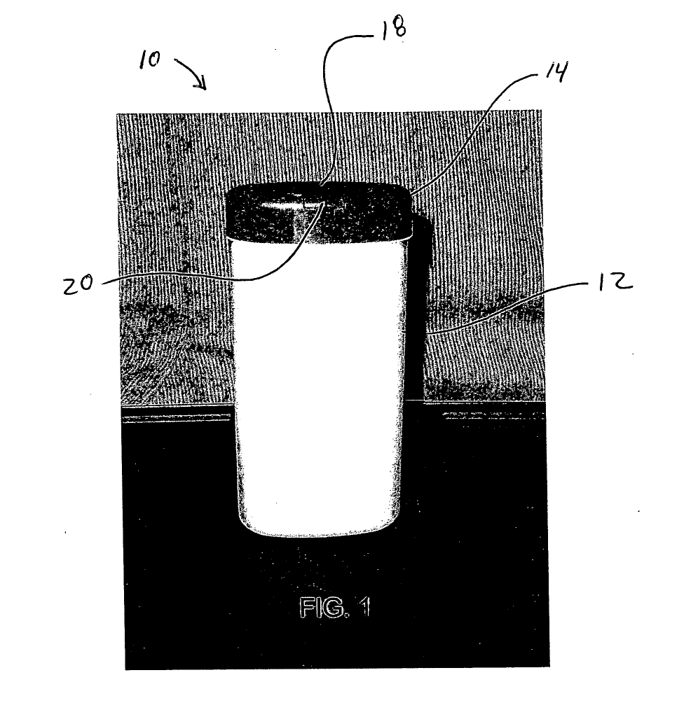

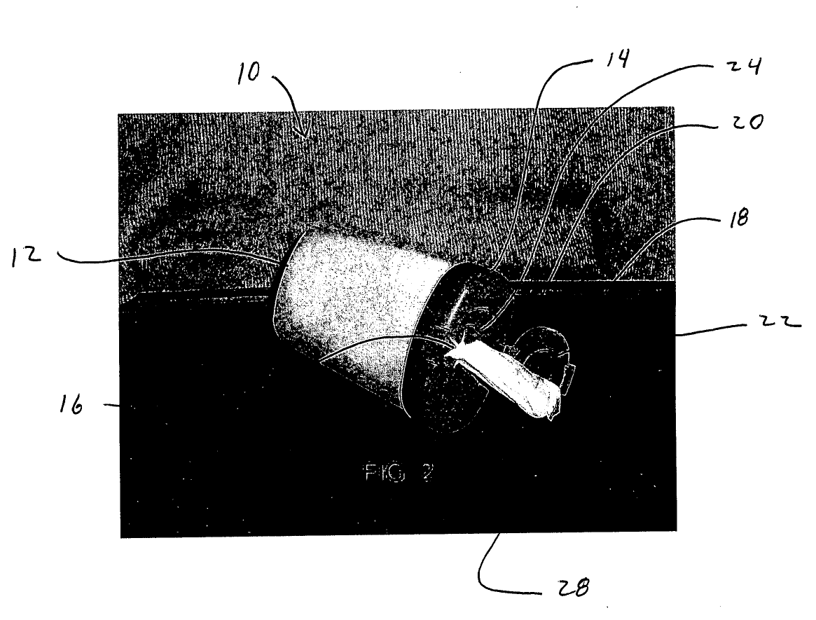

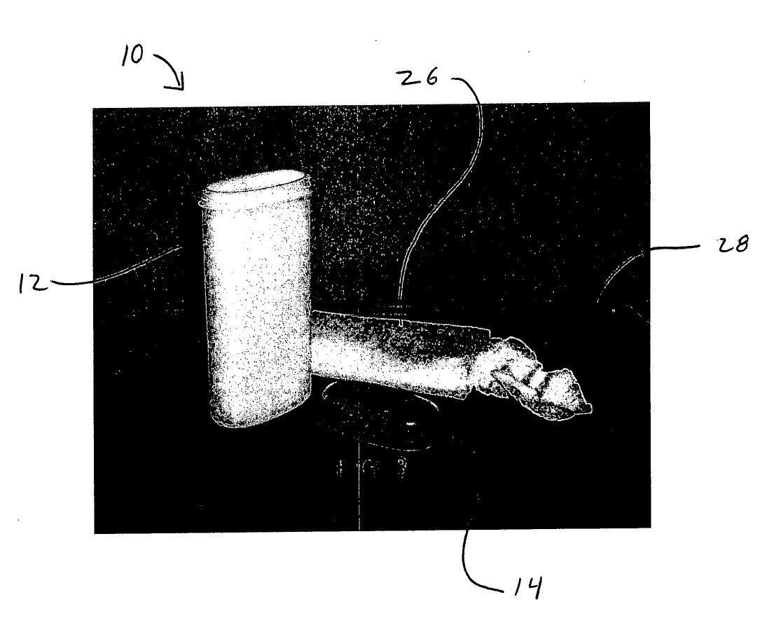

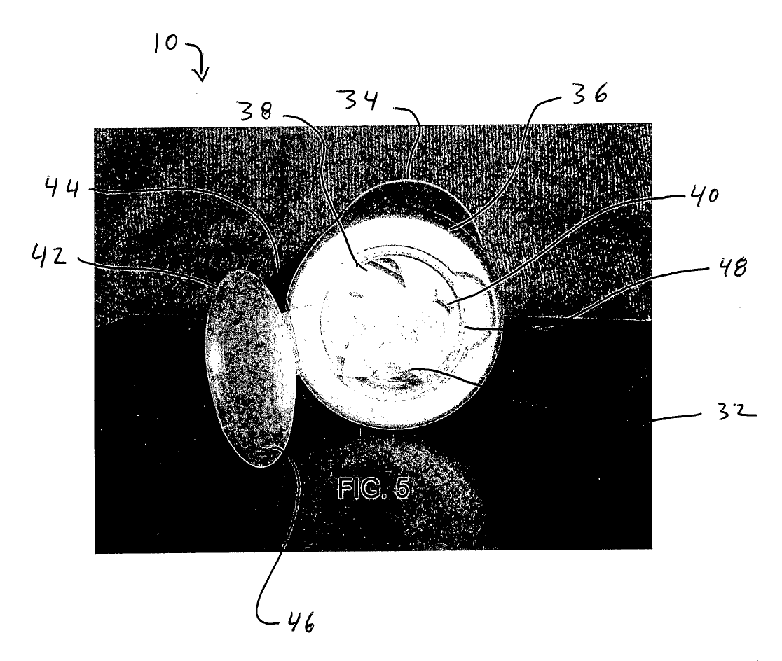

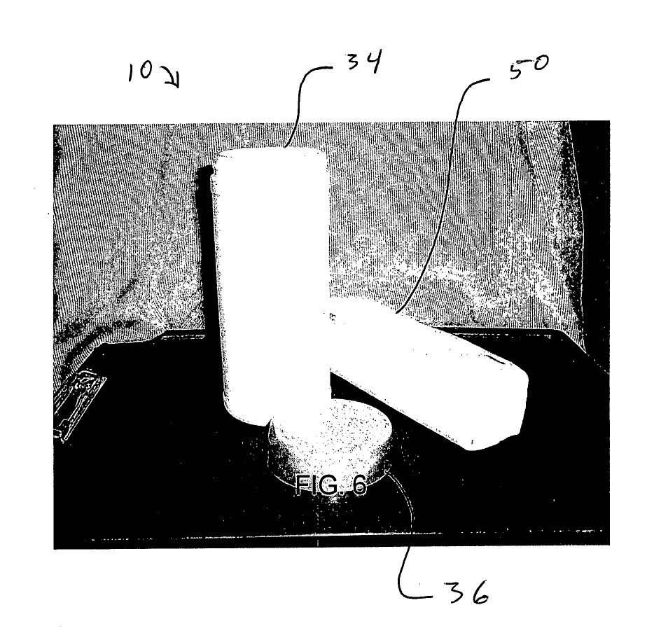

FIGS. 1-6 show examples of animal (pet) wipes having catnip and containers having the animal wipes in accordance with the present invention.

Referring to FIGS. 1-3, a plastic container 10 has a hollow body 12 and a removable top 14. The top 14 is a pull-through top having a dispensing hole 16. The top 14 has a cap 18 on a flexible hinge 20. The cap 18 can cover and close the dispensing hole 16. The cap 18 can be maintained in the closed position by a flange 22 snugly fitting over another flange 24. Other structures can be used to releasably lock the cap 18 closed, such as a snap action lock. A roll 26 of animal wipes having catnip 28 is provided inside of the hollow body 12 of the container 10. The roll 26 of animal wipes having catnip 28 contains a plurality of individual animal wipes having catnip 28 connected together such that an individual animal wipe having catnip 28 can be readily separated from an adjacent animal wipe having catnip 28. For example, two animal wipes having catnip 28 can be joined together along a line of perforations across a continuous sheet of wipes. Adjacent individual animal wipes 28 do not necessarily need to be joined together, but could merely be placed adjacent each other. An individual animal wipe having catnip 28 is dispensed from the container 10 by pulling the animal wipe having catnip 28 out through the dispensing hole 16. An initial portion of the next animal wipe having catnip 28 is also pulled out of the dispensing hole 16 so that it can be easily dispensed when desired. The animal wipe having catnip 28 is positioned close to or in contact with the animal or pet, such as a cat. For example, the cat can be rubbed or wiped with the animal wipe having catnip 28 to deliver the catnip to the cat.

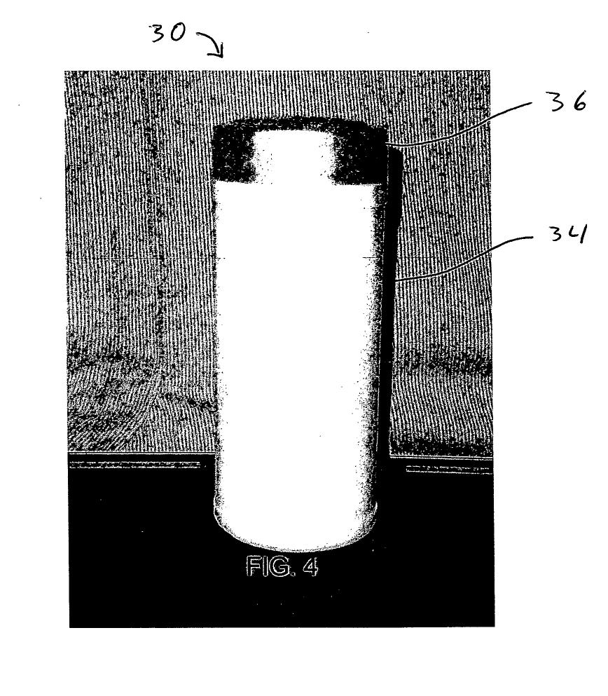

Referring to FIGS. 4-6, another plastic container 30 having animal wipes having catnip 32 is shown. The plastic container 30 has a hollow body 34 and a removable top 36. The top 36 is a pull-through top having a large hole 38 and a small hole 40. The top 36 has a cap 42 on a flexible hinge 44. The cap 42 can cover and close the large and small holes 38, 40. The cap 42 can be maintained in the closed position by a flange 46 snugly fitting over another flange 48. Other structures can be used to releasably lock the cap 42 closed, such as a snap action lock. A roll 50 of animal wipes having catnip 32 is provided inside of the hollow body 34 of the container 30 and dispensed from the container 30 through the large or small holes 38, 40. The large hole 38 can also allow a user to reach into the container 30 with fingers to grab an animal wipe having catnip 32.

The features and advantages of the present invention may be desired, but, are not necessarily required to practice the present invention.

It should be understood that various changes and modifications to the presently preferred embodiments described herein will be apparent to those skilled in the art. Such changes and modifications can be made without departing from the spirit and scope of the present invention and without diminishing its intended advantages.

The invention is claimed as follows:

1. A pet wipe comprising an applicator having a formulation having catnip.

2. The pet wipe of claim 1, wherein the applicator is a towelette.

3. The pet wipe of claim 1, wherein the applicator is a cloth.

4. The pet wipe of claim 1, wherein the catnip is in an amount of about 0.000000001% to about 75% of the formulation carried by the applicator.

5. The pet wipe of claim 4, wherein the amount of the catnip is any numerical range within about 0.000000001% to about 75% of the formulation.

6. The pet wipe of claim 1, wherein the catnip is in an amount of about 0.001% to about 10% of the formulation carried by the applicator.

7. The pet wipe of claim 1, wherein the catnip is in an amount of about 0.5% of the formulation carried by the applicator.

8. The pet wipe of claim 1, wherein the formulation comprises water.

9. The pet wipe of claim 8, wherein the formulation comprises oil.

10. The pet wipe of claim 1, further comprising a container containing the applicator.

11. A method of providing catnip to an animal, comprising wiping the animal with a wipe having a formulation having catnip.

12. The method of claim 11, further comprising dispensing the wipe from a container.

13. A method of making a pet wipe, comprising:
applying a catnip formulation to a wipe;
placing the wipe inside of a container; and
closing the container.

14. The method of claim 13, wherein applying a catnip formulation to a wipe comprises applying a catnip formulation having about 0.000000001% to about 75% catnip.

15. The pet wipe of claim 13, wherein applying a catnip formulation to a wipe comprises applying a catnip formulation having about 0.001% to about 10% catnip.

16. The pet wipe of claim 13, wherein applying a catnip formulation to a wipe comprises applying a catnip formulation having about 0.5% catnip.

17. The pet wipe of claim 13, wherein applying a catnip formulation to a wipe comprises applying a catnip formulation having water.

18. The pet wipe of claim 17, wherein applying a catnip formulation to a wipe comprises applying a catnip formulation having oil.

19. A liquid catnip formulation carried by a wipe.
